(19) United States
(12) Patent Application Publication
(10) Pub. No.: US 2005/0085833 A1
(43) Pub. Date: Apr. 21, 2005

(54) Methods and systems for conjoining tendons, ligaments and the like

(76) Inventor: Tewodros Gedebou, Los Angeles, CA (US)

Correspondence Address: STETINA BRUNDA GARRED & BRUCKER, 75 ENTERPRISE, SUITE 250, ALISO VIEJO, CA, 92656, US

(21) Appl. No.: 10/689,188

(22) Filed: Oct. 20, 2003

(51) Int. Cl. A61B 17/08

(52) U.S. Cl. 606/151

(57) ABSTRACT

Systems and methods for reattaching severed tendons and ligaments are disclosed. According to a preferred embodiment, the system comprises first and second segments a suture or cord that each have at least one anchor mechanism formed along the length thereof. Each respective suture cord segment is axially positioned within respective ones of the severed tendons and, by virtue of the anchor mechanisms formed on such cords, remain securely nested axially therewithin. A fastener member, formed on the ends of the suture cord segment, deposited within the respective ends of the tendon and are coupled to one another to thus cause the tendon to become reattached.

### CROSS-REFERENCE TO RELATED APPLICATIONS

Not Applicable.

### STATEMENT RE: FEDERALLY SPONSORED RESEARCH/DEVELOPMENT

Not Applicable.

### BACKGROUND OF THE INVENTION

Surgical procedures for repairing torn tendons and ligaments are well-known in the art. Essentially, such procedures involve the reattachment of the severed ends of the ligament or tendon by securing the same back together, typically via the surgical placement of sutures tied to the opposed ends of the tendon and ligament.

Such procedure, however, suffers from numerous drawbacks. Most significant of such drawbacks include the precise surgical placement of the sutures across the respective tendons, which must be done with great precision in order to insure proper contact between the opposed ends, and especially such that the same remain in abutment with one another to thus facilitate proper healing. To achieve that end, however, typically requires the use of sophisticated, small-scale surgical instruments that place significant demands on the surgeon. Such procedures become especially problematic to the extent repair of such torn tendons and ligaments are small and/or otherwise difficult to access. In such situations, significant time is needed to necessarily perform such delicate and tedious procedures.

Along these lines, due to the delicate nature by which such surgery is performed, a substantial risk of postoperative complications can arise to the extent mobility of the afflicted tendon is not substantially restricted. In this regard, and as is most prevalent in tendon repair related to the hands and fingers, a substantial risk of rupture occurs to the extent the fingers or hands are not substantially immobilized. Rendering fingers and/or hands immobilized, however, in turn causes stiffness at such afflicted area that can only be overcome by aggressive, competently administered physical therapy.

As such, there is a substantial need in the art for systems and methods that can be rapidly and accurately deployed to facilitate the reattachment of a severed tendon or ligament. There is additionally a need in the art for such a system and method that is relatively easy to deploy and can be performed in a substantially less amount of time and requiring substantially less precision than prior art procedures. There is still further a need in the art for such a system and method that further is operative to securably position the opposed ends of the torn tendon and ligament to the same if not greater degree than prior art surgical procedures and are thus operative to provide increased early postoperative mobilization that substantially minimizes the risk of post surgical rupturing and can thus attain substantially favorable surgical outcomes.

### BRIEF SUMMARY OF THE INVENTION

The present invention specifically addresses and alleviates the above-identified deficiencies in the art. In this regard, the present invention is directed to systems and methods for rapidly and accurately reattaching the opposed severed ends of a ligament or tendon to thus enable the same to heal according to its proper physiological orientation and positioning. According to a preferred embodiment, the system comprises first and second segments of a suture or cord having distal and proximal ends and operative to be implanted axially within dedicated ones of the severed end of the tendon or ligament. Such implantation may be accomplished by depositing a respective segment axially within the tendon via a needle, stylet or other piercing object. To facilitate the ability of the segments to remain implanted axially within such tendon, each respective segment will have formed thereon at least one anchor mechanism. Preferably, each respective segment will have a plurality of anchor mechanisms that are operative to enable each respect suture segment to be advanced axially within the tendon, but resist movement in a rearward direction.

Once each respective segment is axially implanted within a respective one of the severed ends of the tendon, the same are attached to one another to thus cause the opposed ends of the severed tendon to compressively abut one another and assume their proper physiological orientation. To facilitate the ability of the respective segments to become attached to one another, fastener mechanisms formed on the respective proximal ends of such segments will be provided that are interconnectable with one another to thus provide for easy and rapid fixation. Once so conjoined, the opposed ends of the ligament may be bandaged and allowed to heal in due course.

### BRIEF DESCRIPTION OF THE DRAWINGS

These as well as other features of the present invention will become more apparent upon reference to the drawings.

FIG. 1 is a perspective view of a section of bone having a torn tendon extending thereupon.

FIG. 2 is a view of the bone and tendon of FIG. 1, the latter having been reattached via the use of a ligament reattachment mechanism constructed in accordance with a preferred embodiment of the present invention.

FIG. 3 is a partial perspective view of the opposed ends of a severed tendon wherein each respective end of the tendon are implanted with the reattachment system of the present invention.

FIG. 4 is a perspective view of the opposed ends of the tendon depicted in FIG. 3 wherein the same are shown reattached utilizing the system of the present invention.

FIG. 5 is a perspective view of the reattached end of the tendon of FIG. 4 with the same having a bandage radially positioned about the point of severance.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

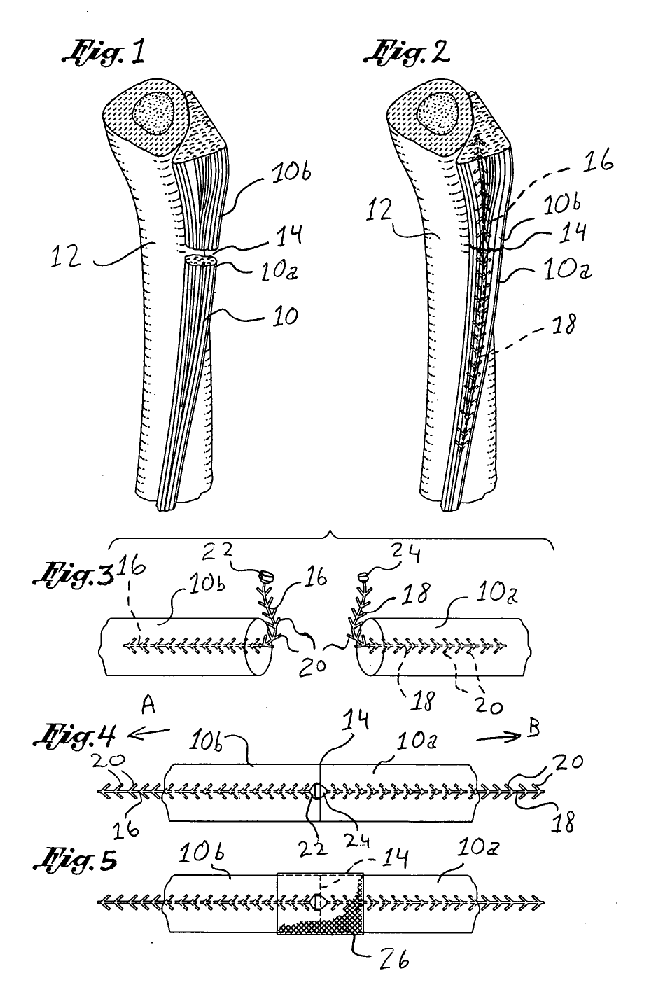

Referring now to the figures, and initially to FIG. 1, there is shown a tendon 10 having a tear formed across a portion thereof to define a first tendon fragment 10a and a second fragment 10b as the same extend along the length of bone 12. In this regard, the cut or tear 14 which defines the respective ligament/tendon portions 10a, 10b is a frequently encountered traumatic event often requiring surgery in order to repair such damage and effectuate proper healing. As discussed in the background, however, such prior art surgical procedures are complicated, tedious and require great precision, and even if performed under the best of circumstances do not guarantee a favorable patient outcome.

To address such drawbacks, there is shown in FIG. 2 the use of a ligament or tendon repair system defined by suture or cord segments 16, 18, that are operative to reattach or conjoin the severed portions 10a, 10b, of the tendon to thus quickly and accurately repair such damage and promote proper healing of such tendon. In this regard, the system of the present invention expressly dispenses with the need to carefully suture the severed ends 10a, 10b back together, especially via the use of complex suture tying techniques.

With respect to the system defined by suture segments 16, 18, as well as the procedure by which the same are used to reattach the severed ends of the tendon 10a. 10b, the same are illustrated in FIGS. 3-5, respectively. As illustrated in FIG. 3, a respective segment 16, 18 will be axially implanted within a respective severed ends of the tendon 10a, 10b, as shown. As illustrated, the distal end of first segment 16 will be axially positioned within tendon segment 10b whereas the distal end of segment 18 will be axially positioned within other respective tendon segment 10a. As will be readily appreciated by those skilled in the art, suture or cord segments 16, 18 will be formed from any of a variety of biocompatible materials known in the art and operative to serve as an implant once surgically positioned within each respective tendon segment.

To facilitate the ability of the suture segments 16, 18 to remain securely positioned axially within the respective tendon segments 10a, 10b, each respective segment 16, 18 will have at least one and preferably a multiplicity of anchor members 20 formed along the length thereof. As illustrated, such anchor members make take the form of a plurality of generally V-shaped or arrowhead-shaped members spaced sequentially along the length of the respective segment 16, 18 and operative to be axially advanced within each respective tendon but yet resist movement when pulled in a rearward direction. To effectuate such placement of such suture or cord segments 16, 18, it is contemplated that the same may be deployed via a variety of techniques well-known in the art, such as through the axial insertion of such sutures via a needle, stylet or other like device. It is likewise contemplated that such suture may be deployed through a cannula or other type of hollow deployment structure that is operative to be axially advanced within each respective tendon segments 10a, 10b and thereafter retracted to thus leave the suture segments 16, 18 with anchor mechanisms 20 formed thereon securely in place.

Formed upon the respective ends of each suture segment 16, 18, will be interconnecting fastener portions 20, 22 that are operative to interconnect with one another, as illustrated in FIG. 4. In this respect, a first fastener 20 will be formed on segment 16 whereas a second fastener member 22 will be formed upon the end of segment 18. Such fastener members 20, 22 will be operative to interconnect with one another to thus create a secure attachment between the suture segments 16, 18, as illustrated in FIG. 4.

By virtue of such interconnection, the respective tendon segments 10a, 10b will thus be reattached or conjoined to assume their natural, unsevered configuration, as shown in FIGS. 2 and 4. In this respect, the respective ends of tendon segments 10a, 10b will be compressively held together at the point of severance 14 by virtue of the interconnection between fastener portions 22, 24. Advantageously, due to the anchoring mechanisms formed along the length of each respective suture 16, 18, such suture segments will thus cause the tendon segments 10a, 10b to remain axially compressed against one another insofar as the anchor mechanisms 20 will resist rearward movement from where the segments 10a, 10b are conjoined. Specifically, the arrangement of anchor mechanisms 20 along suture 16 will prevent segment 10b from moving in the direction indicated by the letter A, whereas anchor member 20 formed along the length of suture 18 will resist movement of tendon segment 10a in the direction indicated by the letter B, as illustrated in FIG. 4. As such, the tendon segments 10a, 10b will thus be caused to remain in a reattached configuration in an extremely secure and accurate manner.

Following the deployment of suture segments 16, 18 such that the tendon segments 10a, 10b are conjoined according to the proper physiological orientation, the procedure is concluded via the placement of bandages or coverings, such as 26 about the point of severance 14 to thus enable the tendon segments 10a, 10b to heal in due course. To facilitate the healing process, it is contemplated that bandage 26 may be fabricated from a thin sheet of biological material to thus reduce adhesion formation and reduce the bulk and fraying of the site of cooptation. Advantageously, such procedure can be performed very rapidly and without the need to perform complex suture tying procedures, as per prior art practices. Moreover, each respective tendon segment 10a, 10b will be maintained in a proper orientation and securely maintain an abutment at the point of severance 14 to thus ensure proper healing, which is known as a difficult yet desirable surgical objective.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention. Along these lines, it is contemplated that the systems for conjoining tendons, ligaments and the like may be configured to externally reconnect the severed ends thereof whereby a covering may be fashioned to be positioned about the severed ends of the severed tendon or ligament and cause the same to be compressively engaged with one another and resist separation when the severed ends are pulled in opposed directions. In this regard, it is contemplated that the systems of the present invention may be configured per a conventional Chinese finger trap fabricated from biocompatible material that may merely require positioning the opposed ends of the severed tendon or ligament to one another and then enabling the tendon/ligament “trap” to hold the opposed severed ends in abutment with one another to thus facilitate proper healing per the systems and methods discussed above.

1. A system for conjoining the severed ends of a tendon or ligament comprising:
a. a first elongate cord having a proximal end and a distal end, said distal end of said cord being operative to be axially implanted within a respective one of said ends of said severed tendon or ligament, said cord having at least one anchor mechanism formed thereon for retaining said cord axially within said severed tendon or ligament;
b. a second elongate cord having a proximal end and a distal end, said distal end of said cord being operative to be axially implanted within the respective other of said ends of said severed tendon or ligament, said cord having at least one anchor mechanism formed thereon for retaining said cord axially within said severed tendon or ligament; and
c. a fastener mechanism for coupling said first and second cords to one another once said cords are implanted and anchored within said severed ends of said tendon or ligament such that said severed ends of said tendon or ligament are operatively retained in abutment with one another.

2. The system of claim 1 wherein said fastener mechanism comprises a first fastener member formed upon said proximal end of said first cord and a second fastener member formed upon said proximal end of said second cord, said first and second fastener members being interconnectable with one another and operative to conjoin said first and second cords.

3. The system of claim 1 wherein said cord comprises a suture.

4. The system of claim 1 wherein said first and second cords are formed from a biocompatible material.

5. The system of claim 1 wherein said at least one anchor mechanism formed upon said first and second cords comprise a plurality of anchor mechanisms.

6. The system of claim 5 wherein said plurality of said anchor mechanisms are formed along the length of said first and second cords.

7. The system of claim 6 wherein said anchor mechanisms comprise a plurality of V-shaped prongs formed along the length of said first and second cords.

8. A method for conjoining the severed ends of a severed tendon comprising the steps:
a. providing a first elongate cord having a proximal end and a distal end, said cord being operative to be axially implanted within a respective one of said ends of said severed tendon or ligament, said cord having at least one anchor mechanism formed thereon for retaining said cord axially within said severed tendon or ligament;
b. providing a second elongate cord having a proximal end and a distal end, said distal end of said cord being operative to be axially implanted within a respective other of said ends of said severed tendon or ligament, said cord having at least one anchor mechanism formed thereon for retaining said cord axially within said severed tendon or ligament; and
c. axially positioning said distal end of said first cord provided in step (a) into a respective one of said severed ends of said tendon such that said anchor mechanism remains implanted therein;
d. positioning said distal end of said second cord provided in step (b) into a respective other of said severed ends of said tendon such that said anchor mechanism remains implanted therein; and
e. securing said first and said second cords to one another such that said severed ends of said tendon are operatively retained in abutment with one another.

9. That method of claim 8 wherein in step (e), said first and second cords are fastened to one another via a fastener.

10. The method of claim 9 wherein said fastener comprises a first fastener member formed upon said proximal end of said first cord and a second fastener member formed upon said proximal end of said second cord, said first and second fastener members being interconnectable with one another.
